(19) United States
(12) Patent Application Publication
(10) Pub. No.: US 2023/0145341 A1
(43) Pub. Date: May 11, 2023

(54) FEED ADDITIVE COMPOSITIONS AND METHODS OF USE

(71) Applicant: PMI Nutrition, LLC, Arden Hills, MN (US)

(72) Inventors: David M. Bravo, Yverdon-les-bains (CH); Karin Berger Büter, Uttwil (CH); Bernhard Büter, Uttwil (CH)

(21) Appl. No.: 17/963,350

(22) Filed: Oct. 11, 2022

Related U.S. Application Data
(60) Provisional application No. 63/255,892, filed on Oct. 14, 2021.

(51) Int. Cl. A61K 36/185; A23K 10/30

(52) U.S. Cl. CPC A61K 36/185; A23K 10/30; A61K 2236/333; A61K 2236/37; A61K 2236/35

(57) ABSTRACT

The present disclosure provides a feed additive for domesticated animals that is capable of stimulating muscle function and health, as well as associated methods for promoting muscle function and health and zootechnical performance in domesticated animals. The animal feed additive provided contains Ribes alpinum, or an extract thereof. The present disclosure also describes methods for improving muscle function and health via the dietary inclusion of Ribes alpinum, or an extract thereof.

### REFERENCE TO RELATED APPLICATION

This application claims the benefit of U.S. Provisional Application No. 63/255,892, filed Oct. 14, 2021, which is herein incorporated by reference in its entirety.

### FIELD OF THE INVENTION

The present disclosure relates to the field of animal feed additives, and more specifically to compositions and methods for improving performance of domesticated animals.

### BACKGROUND OF THE INVENTION

The world population is predicted to double in the next 20 years which doubles the needed quantity of food from animal origin. This is a challenge that was addressed before as in the second part of the 20th century, food from animal origin was already doubled. However, where the challenge is similar, the means to achieve it are profoundly different. Indeed, in the 20th century, the spectacular increase of animal production was made possible by progress in the form of classical nutrition, farm management, genetics and quasi systematic use of antibiotic and chemical compounds. Today, the banned use of antibiotics in animal feed in some countries, a limitation on therapeutic drugs at the farm level in other countries, and an increased use of non-traditional feedstuffs in the farm animal diet globally are undoubtedly changing the interaction between the diet and the farm animal, resulting in a diet that is less nutritionally sound than in the recent past. Even as the need for food from animal origin doubles, food must be produced while respecting animal welfare, as well as taking into account consumers' escalating expectations for environmentally safe and drug-free feed.

Due to the spectacular improvement of genetic potential over the last 50 years, modern farm animals have a very powerful metabolism that is driven by their fast growth. Optimizing this metabolism by providing nutrients and non-nutrients is a critical role of the diet fed to these animals. Progress in feeding, in traditional nutrition or in scientific knowledge have improved performance of these animals. For example, scientific progress in the role of the liver, or more recently the intestine, has promoted new ways of approaching dietary inputs in farm animals. One organ, however, is still under-considered. Skeletal muscles are, obviously, the most valuable part of meat producing animals. Besides, possible roles of the muscles in the development of the animal are not well-appreciated. For example, skeletal muscle uses up to 25-30% of the postprandial glucose, and skeletal muscle health is critical to glucose and energy homeostasis. In humans, improving skeletal muscle metabolic function and insulin sensitivity could therefore have a major impact on overall physiology and improve quality of life. Several factors that are omnipresent in farm animal contexts are known perturbators of skeletal muscle metabolic function, including oxidative stress and chronic inflammation. Imbalance between pro- and anti-inflammatory cytokines has a detrimental effect on muscle health. For instance, elevation in circulating proinflammatory cytokines (e.g., TNFα) evokes insulin resistance and alters glucose homeostasis. This is exacerbated in farm animals which are subject to chronic inflammation (REF) or for which the metabolic function of the skeletal muscles is powerful, making them very susceptible to oxidative stress.

There is therefore a great need in the art for development of feed additives that could supplant the need for antibiotics and other ingredients while improving performance of farm animals in a drug free context. In addition, there is a great need in the art for feed additives for improving muscle health in farm animals.

### SUMMARY

The present disclosure provides, in one embodiment, an animal feed ingredient composition comprising an effective amount of Ribes alpinum, or an extract thereof, wherein the effective amount improves the performance of an animal feed to which the ingredient is added. As used herein, “animal” refers to all animals except humans. Examples of animals are non-ruminants, and ruminants. Ruminant animals include, for example, animals such as sheep, goats, cattle, e.g., beef cattle, dairy cows, cows, and young calves, deer, camel, llama and kangaroo. Non-ruminant animals include mono-gastric animals, e.g., pigs or swine (including, but not limited to, piglets, growing pigs, and sows); poultry such as turkeys, ducks and chickens (including but not limited to broiler chicks, layers); horses (including, but not limited to, hot bloods, cold bloods and warm bloods), fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns). Included among such animals that may find use in accordance with the invention are pets, including, but not limited to, dogs and cats.

In certain embodiments, an animal feed provided herein comprises at least one animal feed component selected from the group consisting of a vitamin, a mineral, a probiotic, an enzyme, a flavoring, an amino acid, a fat, an essential oil, and a preservative. Non-limiting examples of a vitamin include, for example, fat-soluble vitamins including vitamin A, vitamin D3, vitamin E, and vitamin K, e.g., vitamin K3; and water-soluble vitamins including vitamin B12, biotin and choline, vitamin B1, vitamin B2, vitamin B6, niacin, folic acid and panthothenate, e.g., Ca-D-panthothenate, and combinations thereof. Non-limiting examples of a mineral include, for example, calcium, magnesium, potassium and sodium, and trace minerals include boron, cobalt, chloride, chromium, copper, fluoride, iodine, iron, manganese, molybdenum, selenium and zinc. Non-limiting examples of a probiotic include, for example, Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococcus acidilactici, Pediococcus sp., Propionibacterium thoenii, Propionibacterium sp. and Streptococcus sp. or any combination thereof. Non-limiting examples of an enzyme include, for example, acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectinesterase, triacylglycerol lipase, xylanase, beta-xylosidase or any combination thereof. Non-limiting examples of a flavoring include, for example, a floral, berry, nutty, caramel, chocolate, peppery, smoky, cheesy or meaty flavor, mints such as peppermint, citrus flavors such as orange and lemon, artificial vanilla, cinnamon and various fruit flavors. Non-limiting examples of an amino acid include, for example, alanine (Ala; A), arginine (Arg; R), asparagine (Asn; N), aspartic acid (aspartate, Asp; D), cysteine (Cys; C), glutamine (Gln; Q), glutamic acid (glutamate, Glu; E), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y) and valine (Val; V), and any combination thereof. Non-limiting examples of a preservative include, for example, sodium sorbate, potassium sorbate, sodium benzoate and potassium benzoate, and combinations thereof.

In some embodiments, the Ribes alpinum composition is obtained from an extract from a plant or part thereof. In further embodiments, the extract is produced from stems, leaf, flowers, branches, roots or fruits of a plant or part thereof, or any combination thereof. In yet further embodiments, the extract is an aqueous, ethanolic, methanolic, isopropanolic, ethylacetate, acetonic, or hexane extract, or mixtures thereof, or a supercritical CO₂ extract.

In other embodiments of a composition provided herein, the Ribes alpinum, or an extract thereof, comprise between about 5% and 100% of the composition, including, for example, at least about 10% of the composition. In other embodiments, Ribes alpinum, or an extract thereof, in said composition comprise at least 10% of said composition.

In still other embodiments, the present disclosure provides an animal feed comprising an animal feed ingredient composition described herein comprising an effective amount of Ribes alpinum, or an extract thereof, in an amount effective to improve zootechnical performance in an animal fed the animal feed relative to a control feed lacking said animal feed ingredient composition. In certain embodiments, the animal feed is a chicken feed, pig feed, dairy cow feed, or beef cattle feed. In specific embodiments, the improved zootechnical performance is improved muscle function and health or increased average daily weight gain, increased feed efficiency, or decreased feed conversion ratio.

The term “feed conversion ratio” as used herein refers the amount of feed fed to an animal to increase the weight of the animal by a specified amount. An improved feed conversion ratio means a lower or decreased feed conversion ratio. By “lower or decreased feed conversion ratio” or “improved feed conversion ratio” it is meant that the use of a feed additive composition in feed results in a lower amount of feed being required to be fed to an animal to increase the weight of the animal by a specified amount compared to the amount of feed required to increase the weight of the animal by the same amount when the feed does not comprise the feed ingredient or additive composition.

The term “feed efficiency” refers to the amount of weight gain per unit of feed when the animal is fed ad-libitum or a specified amount of food during a period of time. By “increased feed efficiency” it is meant that the use of a feed additive composition according the present invention in feed results in an increased weight gain per unit of feed intake compared with an animal fed without the feed ingredient or additive composition being present.

In various embodiments, the Ribes alpinum, or an extract thereof, is present in an amount of from about 3 g to about 100 g to about 200 g or about 500 g per metric ton of animal feed. In certain embodiments the Ribes alpinum, or an extract thereof, is present in an amount of about 100 mg per kg of animal feed.

In still another embodiment, the present disclosure provides a method of improving the performance of an animal feed comprising adding an effective amount of a composition comprising Ribes alpinum, or an extract thereof, as described herein, to the animal feed. In certain embodiments, the improved performance comprises improved muscle health and performance, altered feed intake, increased average daily weight gain, increased feed efficiency, decreased feed conversion ratio or increased milk yield in an animal fed said animal feed relative to a control animal feed to which the animal feed ingredient composition has not been added. In various embodiments, the animal feed is chicken feed, pig feed, dairy cow feed, or beef cattle feed.

In still yet another embodiment, the present disclosure also provides a method of improving the zootechnical performance of an animal comprising feeding to the animal an animal feed comprising an animal feed ingredient composition comprising an effective amount of Ribes alpinum, or an extract thereof, in an amount effective to improve zootechnical performance in an animal fed the animal feed relative to a control feed lacking said animal feed ingredient composition. In some embodiments, the animal is a chicken, pig, dairy cow or beef cattle. In other embodiments, the improved zootechnical performance is improved muscle health and performance, altered feed intake, increased average daily weight gain, increased feed efficiency, decreased feed conversion ratio or increased milk yield.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 presents the concentration in ellagic acid of several Rubus and Ribes plants. The data show the average concentration of ellagic acid in several plants and its standard deviation.

FIG. 2 shows the muscle cell counts of C. elegans for the control treatment and the Ribes alpinum leaf extract treatment. The Ribes alpinum extract provided 300 μg/ml containing 0.0017 μg/ml of ellagitannins, expressed as ellagic acid after hydrolysis. Bars with different letters are significantly different (P<0.01).

FIG. 3 shows the C. elegans thrashing 7 days post L4 stage for the control treatment and the Ribes alpinum leaf extract treatment. The Ribes alpinum extract provided 300 μg/ml containing 0.0017 μg/ml of ellagitannins, expressed as ellagic acid after hydrolysis. Bars with different letters are significantly different (P<0.02).

FIG. 4 shows that the inclusion of 300 μg/mL of Ribes alpinum extract stimulated protein formation and muscle cell proliferation in C2C12 cells in vitro. The Y axis shows the protein content used as an estimate of muscle cell proliferation in percent of the control. The black bar is the control without addition of plant extract; the hatched bar is the Ribes alpinum extract treatment. Standard deviations are shown on the bars.

FIG. 5 shows that the addition of increasing doses of Ribes alpinum extract increased glucose uptake by the C2C12 muscle cell in vitro. They axis is the response of the plant treatment (hatched bar) compared to the control (black bar). Standard deviations are shown on the bars.

### DETAILED DESCRIPTION

The present disclosure provides a feed additive for domesticated animals that is capable of stimulating muscle function and health, as well as associated methods for promoting muscle function and health and zootechnical performance in domesticated animals. The animal feed additive provided herein contains Ribes alpinum, or an extract thereof. The present disclosure also describes methods for improving muscle function and health via the dietary inclusion of Ribes alpinum, or an extract thereof.

Ellagitannins are hydrolysable polyphenols with beneficial effects on human health. In the gastrointestinal tract, ellagitannins are hydrolyzed to ellagic acid, which is then metabolized to urolithins by colon microbiota. This will be denoted as ellagic acid/urolithins in the present specification. Ellagic acid/urolithins show various health promoting effects, i.e., anti-proliferation in cancer cell models, anti-inflammation, optimization of lipid metabolism and induction of autophagy. Animal studies have shown their positive effects on mitochondria and muscle function.

The present disclosure provides example embodiments of a novel composition to be included in the diet of farm animal to increase protein synthesis, muscle mass, and/or muscle strength. The examples describe the inclusion in the food of animals of a plant, Ribes alpinum, or an extract thereof, with the intention of improving performance, muscle growth and muscle health.

Ellagic acid/urolithins is known to have effect on muscle growth. For example, an experiment with urolithins in a C. elegans assay showed that a concentration of 50 μM (11.4 μg/ml) is needed to obtain a positive effect on muscle function. Put another away, a concentration of 11.4 μg/mL of urolithins is required to trigger the muscle function response. Compositions comprising urolithin with a source of protein or a medium chain triglyceride or nicotinamide riboside, respectively, have been suggested to treat muscle-related pathological conditions. Composition including urolithin have also been suggested for increasing muscle cell size. Taken together, it is clear that ellagic acid/urolithins promote muscle function and muscle growth.

Urolithins production can be triggered by including in the diet consumed by a subject a dietary source of ellagitannin which will be digested to ellagic acid and later fermented into urolithins. A well know dietary source of ellagitannin is fruits, nuts, leaves and seeds of certain plant species. For instance, high contents are found, for example, in pomegranate, and berries belonging to Rubus and Ribes species. Based on the results summarized above, a minimum of 11.4 mg/mL of urolithin is required to promote muscle cell health and function.

However, the present disclosure surprisingly found including a plant that does not contain or contains very low amount of ellagic acid/urolithin.

### Animal Feed Components

Provided herein are novel animal feed compositions with improved performance as a result of comprising the animal feed ingredient composition described herein. Different domesticated animals have different feed requirements. For example, the main ingredients in chicken feed are generally cereals, including, but not limited to, wheat, corn, sorghum, oats, barley or rye, protein, which can come from oilseed meals, and fat or oil. However, other ingredients can be added to chicken feed, including, but not limited to, a source of calcium, salts, minerals, probiotics, vitamins, amino acids, flavorings and preservatives. An exemplary chicken feed for chicks and pullets includes protein, lysine, methionine, fat, fiber, calcium, phosphorous, NaCl, manganese, vitamin A and vitamin E.

The main ingredients in pig feed are generally cereals, including, but not limited to, rice bran, broken rice, and corn, protein, which can come from oilseed meals such as alfalfa meal, or soybean meal, minerals and vitamins. However, other ingredients can be added to pig feed, including, but not limited to, a source of calcium, salts, minerals, probiotics, vitamins, amino acids, flavorings and preservatives. An exemplary pig feed includes an energy source (cereals), protein, vitamins, minerals, fiber, prebiotics and botanicals.

The main ingredients in dairy cattle feed are generally cereals, protein, which can come from oilseed meal such as cottonseed meal or soybean meal, sugar and fat. However, other ingredients can be added to dairy cattle feed, including, but not limited to, fiber, a source of calcium, salts, minerals, probiotics, vitamins, amino acids, flavorings and preservatives.

The main ingredients in beef cattle feed are generally cereals, including, but not limited to, wheat, corn, sorghum, oats, barley or rice, and protein, which can come from de-oiled rice bran, rice polish, wheat bran or corn bran. However, other ingredients can be added to beef cattle feed, including, but not limited to, fiber, fat, salts, minerals, probiotics, vitamins, amino acids, flavorings and preservatives.

The main ingredients in sheep feed are generally cereals, such as alfalfa and corn, vitamins, such as vitamin A, vitamin D and vitamin E, selenium, mineral salts and phosphorous. However, other ingredients can be added to sheep feed, including, but not limited to, fat, probiotics, amino acids, flavorings and preservatives. An exemplary sheep feed for mature ewes and rams includes cereals, protein, fat, fiber, calcium, ammonium chloride, phosphorous, NaCl, selenium and vitamin A.

The main ingredients in goat feed are generally cereals, such as hay, alfalfa, barley corn and oats, protein, which can come from distilled grains and meals, fat, fiber and minerals such as calcium, phosphorous, NaCl, copper, selenium, and vitamins such as vitamin A, vitamin D and vitamin E. However, other ingredients can be added to goat feed, including, but not limited to, probiotics, amino acids, flavorings and preservatives. An exemplary goat feed includes grain products, protein, fat, fiber, acid detergent fiber and calcium, phosphorous, NaCl, as well as copper, selenium, and vitamins such as vitamin A, vitamin D and vitamin E.

As described herein, the performance of any such animal feeds may be improved by the addition of the animal feed ingredient provided by the present disclosure. The amount added may be optimized depending upon the type of feed, animal physiology, conditions under which the animal being fed is raised, and other conditions as will be understood to those of skill in the art according to the teachings of the present disclosure.

### EXAMPLES

### Example 1

The leaves of various berry varieties were analyzed by HPLC for their ellagic acid content. The leaf samples were extracted to obtain the ellagitannins, which were then acid-hydrolyzed resulting in ellagic acid. The ellagic acid content was determined by a HPLC method using ellagic acid as external standard.

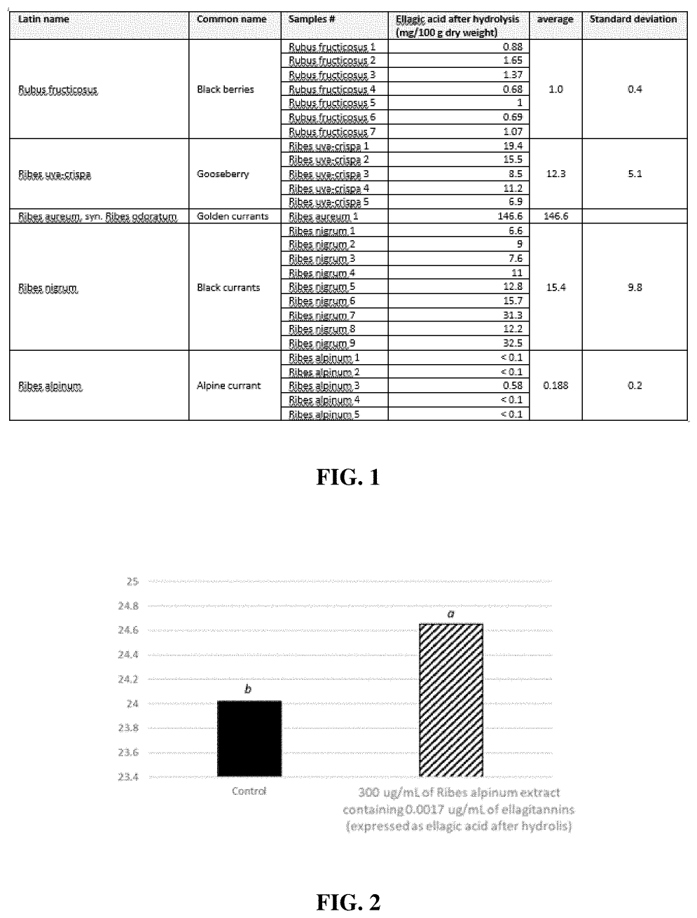

FIG. 1 gives an overview on the ellagic acid content, after hydrolyzation. The ellagic acid content in leaves of different Rubus fruticosus varieties varied from 0.68 to 1.65 mg/100 g dry weight. In gooseberry leaves the content varied from 6.9 to 19.4 mg/100 g. Higher amounts were seen in Ribes nigrum leaf samples, where the content varied from 6.6 to 32.5 mg/100 g. The absolute highest content of ellagic acid, i.e., 146.6 mg/100 g, was detected in a sample of Ribes aureum, resp. R. odoratum (syn.). The lowest ellagic acid content was found for Ribes alpinum, i.e., 0.188 mg/100 g with 4 samples out of 5 only having traces of ellagic acid. This analysis confirmed that Ribes alpinum, although a Ribes plant, contains only traces of ellagic acid.

### Example 2

Ribes alpinum was tested for its potential effect on muscle growth in a C. elegans test model. The Ribes alpinum leaf extract was provided as 300 μg/mL solutions in 50% ethanol. This extract contained 0.58 μg/100 mg of ellagitannins (expressed as ellagic acid after hydrolysis). Assuming that ellagic acid is totally converted into urolithins, the urolithin concentration in the C. elegans that was treated with the Ribes alpinum extract is only 0.0017 μg/ml.

For all experiments, nematode growth media (NGM) supplemented with the extracts were prepared as follows: A single aliquot of sample was defrosted in a water bath at 45° C. for 15 minutes. The fully dissolved sample was filtered through a 0.2 μm sterile filter with a cellulose acetate membrane into a clean 1.5 mL Eppendorf tube. Each extract was added to NGM to a final concentration of 300 μg/mL and poured into petri dishes. The relevant controls included a negative control and ethanol solvent controls, at 0.15% and 0.075%. For all conditions, NGM was prepared that also contained 100 μM 5-fluoro-T-deoxyuridine (FuDR) to prevent hatching of offspring. Age-synchronized worms were placed onto the compound supplemented NGM from L1 to L4 stage and assessed for mobility and the number of muscle cells. When L4, worms were transferred to compound plates. The mobility and muscle morphology of animals were further assessed at 7 days post-L4 (after the reproductive period has finished).

The muscle cells proliferation was assessed. The worm strain AW306, a wild type animal that expresses a nuclear marker for body wall muscle nuclei, was used. Worms were grown from L1 to L4 on each of the compound plates. When worms reached the L4 stage, the number of muscle cell nuclei was counted in the right ventral quadrant (n=40).

The mobility of the worms was assessed. Wild type worms (strain, N2) from each condition (20-25 worms), were placed into M9 buffer and videos were taken using a Leica S8aP0 binocular microscope with a Leica DMC2900 camera and the LAS v4.12 software. The movies were analyzed using ImageJ v1.53. The number of body bends was quantified using the wrMTrck plugin (build 110622) for ImageJ. The body bends per minute (BBPM) were calculated and graphs generated using GraphPad Prism displaying the average and standard error of the mean, with each dot representing a single worm counted.

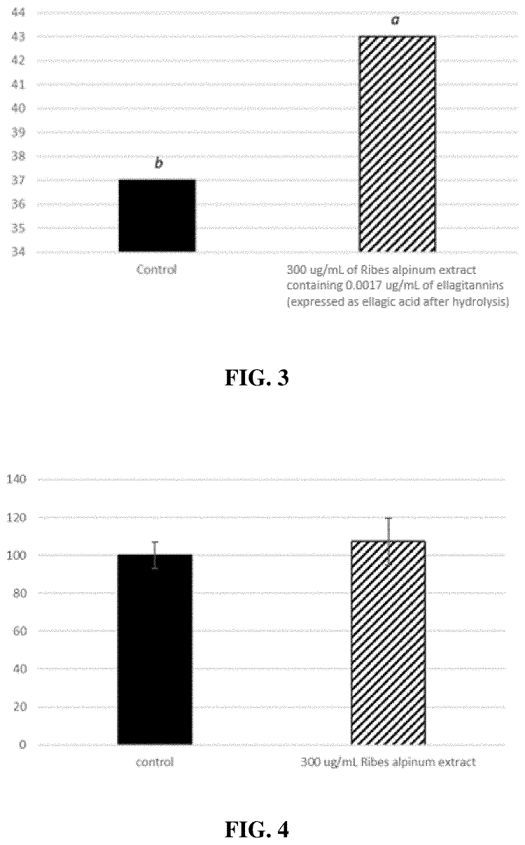

The following results were observed. Ribes alpinum extract, containing 0.0017 μg/ml ellagitannins (expressed as ellagic acid after hydrolysis) showed a positive effect on muscle growth by increasing the muscle proliferation at L4 stage in C. elegans (FIG. 2). Ribes alpinum extract, providing 0.0017 μg/ml ellagitannins (expressed as ellagic acid after hydrolysis) showed a positive effect on muscle function by increasing muscle thrashing in C. elegans 7 days post L4 (FIG. 3). Considering that the minimum concentration of urolithin to produce a muscle function response is 11.4 mg/mL of urolithin, as detailed above, the inclusion of ellagic acid/urolithin is 1.49123E-07 smaller than the minimum dose. Such a very low dose of ellagic acid/urolithin cannot promote muscle growth. Surprisingly, 0.3 mg of Ribes alpinum leaf extract, supplying 0.0017 μg/mL of ellagic acid/urolithin showed positive effect on muscle growth. It can be concluded that the effect on muscle growth of Ribes alpinum is not caused by ellagitannins.

### Example 3

This example evaluated the effect of Ribes alpinum extract on the physiology of the muscle cell in a cell-based bioassay.

The bioassay was performed with a mouse myoblast cell line called C2C12. Ribes alpinum extract was tested at different concentrations in triplicates. Two outcomes were measured. First, the muscle cell proliferation assay aimed at investigating the influence of Ribes alpinum extract, applying 300 μg/ml, on muscle cell proliferation and growth. The determination of the protein content was used as a mean to identify cell proliferation in the applied in vitro model. The first supplementation of the C2C12 mouse myoblast cell line with Ribes alpinum extract was performed 24 hours after seeding. After five days, differentiation of the cells was initiated. The second supplementation with Ribes alpinum extract was performed 24 hours after the beginning of the differentiation. At day 3 of the differentiation phase, the last supplementation with Ribes alpinum extract was performed. Cells were lysed 7 days after the beginning of the differentiation and the protein content was determined using a Bradford assay. A medium control was performed with each batch. The assays were carried out in triplicate. Second, the glucose uptake assay aimed at investigating the influence of Ribes alpinum extract on glucose uptake in muscle cells. C2C12 cells were seeded and grown to confluency (3 days). Cells were differentiated for 4 days to form myotubes and were then treated with Ribes alpinum extract for 24 hours. After supplementation, glucose was depleted for 24 hours followed by serum depletion overnight. After washing, a fluorescent glucose analog for monitoring glucose uptake into living cells called 2-NBDG (2-(N-(7-nitrobenz-2-oxa-1,3-diazo14-yl)-amino)-2-deoxyglucose) was added to the medium. After 30 minutes fluorescence was measured after washing and lysing the cells. The assay was carried out in n=6.

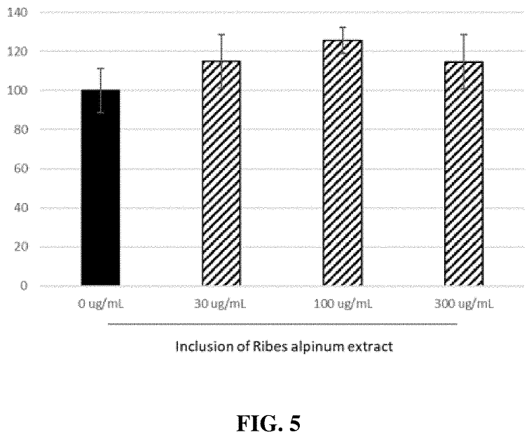

The results indicated that 300 μg/mL of Ribes alpinum extract increased muscle cell proliferation (FIG. 4) and that 30 μg/ml increased glucose uptake (FIG. 5). These results confirmed the fact that the muscle growth response of Ribes alpinum is not mediated by ellagic acid/urolithin. In this example, a cell-based assay with C2C12 cells was used. It is known that only gut residing bacteria can convert ellagic acid into urolithin (REF). It is also well-known that this cell-based bioassay does not allow an effect of components that require a biotransformation by microbes to be seen. Therefore the effect of Ribes alpinum extract on muscle cell proliferation and function is not dependent on ellagic acid, which needs to be transformed into urolithin by gut microbes.

Taken together, the inclusion of small amounts of Ribes alpinum in the diet of farm animal can be used to promote muscle health and function. Ribes alpinum can in certain embodiments be included in the diet up to 500 g of plant per metric ton of feed, while in other embodiments the dose is between 100 and 200 g of plant per metric ton of feed.

To facilitate the understanding of this disclosure, a number of terms are defined below. Terms defined herein have meanings as commonly understood by a person of ordinary skill in the areas relevant to the present disclosure. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. For example, all of the disclosed components of the preferred and alternative embodiments are interchangeable providing disclosure herein of many systems having combinations of all the preferred and alternative embodiment components. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims. All such similar substitutes and modifications apparent to those skilled in the art are deemed to be within the spirit, scope and concept of the disclosure as defined by the appended claims.

1. An animal feed ingredient composition comprising an effective amount of Ribes alpinum, or an extract thereof, wherein the effective amount improves the performance of an animal feed to which the ingredient is added.

2. The composition of claim 1 further defined as comprising at least one animal feed component selected from the group consisting of a vitamin, mineral, probiotic, enzyme, flavoring, amino acid, fat, essential oil, and preservative.

3. The composition of claim 1, wherein the extract is produced from a stem, leaf, flower, root, or fruit of a Ribes alpinum plant or part thereof, or any combination thereof.

4. The composition of claim 1, wherein the extract is an aqueous, ethanolic, methanolic, isopropanolic, ethylacetate, acetonic, hexane, or a mixture thereof, or a supercritical CO₂ extract.

5. The composition of claim 1, wherein the Ribes alpinum or extract thereof comprises between about 3 g/metric ton and about 500 g/metric ton of the composition.

6. The composition of claim 5, wherein the Ribes alpinum or extract thereof comprises between about 9 g/metric ton and about 90 g/metric ton of the composition.

7. An animal feed comprising the animal feed ingredient composition of claim 1 in an amount effective to improve zootechnical performance in an animal fed the animal feed relative to a control feed lacking the animal feed ingredient composition.

8. The animal feed of claim 7, wherein the animal feed is chicken feed, pig feed, dairy cow feed, or beef cattle feed.

9. The animal feed of claim 7, wherein the improved zootechnical performance is improved muscle function or health, altered feed intake, increased average daily weight gain, increased feed efficiency, decreased feed conversion ratio, or increased milk yield.

10. The animal feed of claim 7, wherein the Ribes alpinum or extract thereof is present in an amount of from about 3 g to about 500 g per metric ton of animal feed.

11. A method of improving the performance of an animal feed, comprising adding an effective amount of the composition of claim 1 to the animal feed.

12. The method of claim 11, wherein the improved performance comprises improved muscle function or health, altered feed intake, increased average daily weight gain, increased feed efficiency, decreased feed conversion ratio, or increased milk yield in an animal fed the animal feed relative to a control animal feed to which the animal feed ingredient composition has not been added.

13. The method of claim 11, wherein the animal feed is chicken feed, pig feed, dairy cow feed, or beef cattle feed.

14. The method of claim 11, wherein the Ribes alpinum or extract thereof is present in an amount of from about 3 g to about 500 g per metric ton of animal feed.

15. A method of improving the zootechnical performance of an animal comprising feeding the animal feed of claim 7 to the animal.

16. The method of claim 15, wherein the animal is a chicken, pig, dairy cow, or head of beef cattle.

17. The method of claim 15, wherein the improved zootechnical performance is improved muscle function or health, altered feed intake, increased average daily weight gain, increased feed efficiency, decreased feed conversion ratio, or increased milk yield.

18. The method of claim 15, wherein the animal feed comprises the Ribes alpinum or extract thereof in an amount of from about 3 g to about 500 g per metric ton of animal feed.
